(19) United States
(12) Patent Application Publication
(10) Pub. No.: US 2026/0199600 A1
(43) Pub. Date: Jul. 16, 2026

(54) DEVICES, SYSTEMS, AND METHODS FOR CLOSED- AND SEMI-CLOSED-LOOP OPERATION OF INFUSION PUMPS

(71) Applicant: Tandem Diabetes Care, Inc., San Diego, CA (US)

(72) Inventors: Thomas Ulrich, San Diego, CA (US); Nicholas Sherer, Escondido, CA (US); Ryan Cardenas, San Diego, CA (US); Ricardo Rueda, San Diego, CA (US); Micah Stephens, San Diego, CA (US); Colin Van Meter, San Diego, CA (US); Peter Zhao, San Diego, CA (US); Patrick Mulvihill, San Diego, CA (US); John Corbett, San Diego, CA (US); Katherine Vyvy Tran, San Diego, CA (US); Dulguun Gantulga, San Diego, CA (US); Peter Briggs, San Diego, CA (US)

(21) Appl. No.: 19/134,978

(22) PCT Filed: Dec. 1, 2023
(86) PCT No.: PCT/US2023/082084

§ 371 Date: Jun. 2, 2025

Related U.S. Application Data
(60) Provisional application No. 63/429,407, filed on Dec. 1, 2022.

(51) Int. Cl. A61M 5/172; G16H 20/13

(52) U.S. Cl. CPC A61M 5/1723; G16H 20/13; A61M 2205/502; A61M 2230/201

(57) ABSTRACT

A system for operation of an infusion pump may include a device configured to generate dosing functions for delivery of a medicament via an infusion pump. The system may also include the infusion pump, where the infusion pump is configured to receive the dosing functions from the aforenoted device, select one of the dosing functions based on characteristics of a user, and deliver a dose of the medicament according to the selected dosing function. Another system may include an infusion pump and an electronic device. The electronic device can be configured to provide user information to a neural network, receive dosing instructions from the neural network, and determine whether the dosing instructions satisfy a guardrail threshold. If the guardrail threshold is satisfied, then the electronic device can cause the infusion pump to deliver an amount of a medicament according to the dosing instructions.

### CROSS-REFERENCE TO RELATED APPLICATION

The Present application claims priority to U.S. Provisional Patent App. No. 63/429,407, filed Dec. 1, 2022, the disclosure of which is incorporated in its entirety herein.

### TECHNICAL FIELD

The present disclosure relates, generally, to ambulatory infusion pumps and, more specifically, to closed- or semi-closed-loop operation of infusion pumps.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent App. Pub. Nos. 2013/0324928 and 2013/0053816, as well as U.S. Pat. Nos. 8,287,495, 8,573,027, 8,986,253, and 9,381,297, each of which is incorporated herein by reference in its entirety.

Ambulatory infusion pumps for delivering insulin or other medicaments can be used in conjunction with blood glucose monitoring systems, such as blood glucose meters (BGMs) and continuous glucose monitors (CGMs). A CGM provides a substantially continuous estimated blood glucose level through a transcutaneous sensor that estimates blood analyte levels, such as blood glucose levels, via the patient's interstitial fluid CGM systems typically consist of a transcutaneously placed sensor, a transmitter, and a monitor.

Ambulatory infusion pumps typically allow the patient or caregiver to adjust the amount of insulin or other medicament delivered, by a basal rate or a bolus, based on blood glucose data obtained by a BGM or a CGM, and in some cases include the capability to automatically adjust such medicament delivery. Some ambulatory infusion pumps may include the capability to interface with a BGM or CGM such as, e.g., by receiving measured or estimated blood glucose levels and automatically adjusting or prompting the user to adjust the level of medicament being administered or planned for administration or, in cases of abnormally low blood glucose readings, reducing or automatically temporarily ceasing or prompting the user temporarily to cease or reduce insulin administration. These portable pumps may incorporate a BGM or CGM within the hardware of the pump or may communicate with a dedicated BGM or CGM via wired or wireless data communication protocols, directly and/or via a device such as a smartphone. One example of integration of infusion pumps with CGM devices is described in U.S. Patent App. Pub. No. 2014/0276419, which is hereby incorporated by reference herein.

As noted above, insulin or other medicament dosing by basal rate and/or bolus techniques could automatically be provided by a pump based on readings received into the pump from a CGM device that is, e.g., external to the portable insulin pump or integrated with the pump as a pump-CGM system in a closed-loop or semi-closed-loop fashion. With respect to insulin delivery, some systems including this feature can be referred to as artificial pancreas systems because the systems serve to mimic biological functions of the pancreas for patients with diabetes. Such systems are also referred to as automated insulin delivery (AID) systems.

Ambulatory infusion pumps have relatively low memory and processing capabilities, and therefore corresponding computational power, as well as battery life, compared to, e.g., a computer system. As such, current AID systems employ relatively simple algorithms that calculate insulin doses in a manner consistent with the limited capabilities of such pumps. For example, current systems may employ a single control variable and a single target (or target range) because they do not possess the computing capability and/or battery life to employ more complex algorithms.

### SUMMARY

Embodiments of the present disclosure provide apparatuses and methods for automated insulin delivery.

A system for operation of an infusion pump includes a dosing function device and an infusion pump. The dosing function device is configured to generate a predictive model to simulate the effects of dosing decisions over time. The dosing function device is also configured to determine termination states based on the predictive model, where each of the termination states represents an outcome following a sequence of dosing decisions over a finite time horizon. Additionally, the dosing function device is configured to apply a cost function to each dose in the sequence of dosing decisions such that each termination state has an associated cumulative cost. Further, the dosing function device is configured to determine dosing functions, where each of the dosing functions represents the cumulative cost associated with each termination state. The infusion pump is configured to receive the dosing functions from the dosing function device. The infusion pump is also configured to select one of the dosing functions based on one or more characteristics of a user. Additionally, the infusion pump is configured to deliver a dose of a medicament to the user according to the selected dosing function.

An infusion pump includes a processor and a non-transitory, computer-readable medium storing instructions which, when executed by the processor, cause the infusion pump to perform operations. The operations include receiving a set of fundamental dosing functions from a dosing function device, where each of the fundamental dosing functions represents a cumulative cost associated with each of a plurality of termination states, and where each of the plurality of termination states represents a respective outcome following a sequence of dosing decisions over a finite time horizon. The operations also include estimating a matching factor based on a basal rate and a correction factor. Additionally, the operations include selecting a fundamental dosing function from the set of fundamental dosing functions based on the matching factor and a target glucose. Further, the operations include estimating a scaling factor based at least on a basal rate of the selected fundamental dosing function. Moreover, the operations include determining a derived dosing function by multiplying the selected fundamental dosing function by the scaling factor. Furthermore, the operations include delivering a dose of medicament to a user according to the derived dosing function.

A computer-implemented method for operation of an infusion pump includes receiving a set of fundamental dosing functions at the infusion pump and from a dosing function device. Each of the fundamental dosing functions represents a cumulative cost associated with each of a plurality of termination states, where each of the plurality of termination states represents a respective outcome following a sequence of dosing decisions over a finite time horizon. The method also includes estimating, at the infusion pump, a matching factor based on a basal rate and a correction factor. Additionally, the method includes selecting, at the infusion pump, a fundamental dosing function from the set of fundamental dosing functions based on the matching factor and a target glucose. Further, the method includes estimating, at the infusion pump, a scaling factor based at least on a basal rate of the selected fundamental dosing function. Moreover, the method includes determining, at the infusion pump, a derived dosing function by multiplying the selected fundamental dosing function by the scaling factor. Furthermore, the method includes delivering a dose of medicament to a user via the infusion pump and according to the derived dosing function.

A system for neural-network-assisted operation of an infusion pump includes an infusion pump and an electronic device. The infusion pump is configured to deliver a medicament to a user. The electronic device is configured to receive user information regarding the user. The electronic device is also configured to provide the user information to a neural network. Additionally, the electronic device is configured to receiving dosing instructions from the neural network responsive to providing the user information to the neural network. Further, the electronic device is configured to compare the dosing instructions against a guardrail threshold. Moreover, the electronic device is configured to, responsive to determining that the dosing instructions satisfy the guardrail threshold, cause the infusion pump to deliver an amount of the medicament to the user according to the dosing instructions. Furthermore, the electronic device can be configured to, responsive to determining that the dosing instructions do not satisfy the guardrail threshold, cause the infusion pump to deliver an amount of the medicament corresponding to a maximum or a minimum threshold of the guardrail threshold nearest the dosing instructions.

A non-transitory, computer-readable medium stores instructions which, when executed by a processor of an electronic device, cause the electronic device to perform operations. The operations include receiving user information regarding a user. The operations also include providing the user information to a neural network. Additionally, the operations include receiving dosing instructions from the neural network responsive to providing the user information to the neural network. Further, the operations include comparing the dosing instructions against a guardrail threshold. Moreover, the operations include, responsive to determining that the dosing instructions satisfy the guardrail threshold, causing an infusion pump to deliver an amount of a medicament to the user according to the dosing instructions.

A computer-implemented method for neural-network-assisted operation of an infusion pump includes receiving user information regarding a user. The method also includes providing the user information to a neural network. Additionally, the method includes receiving dosing instructions from the neural network responsive to providing the user information to the neural network. Further, the method includes comparing the dosing instructions against a guardrail threshold. Moreover, the method includes, responsive to determining that the dosing instructions satisfy the guardrail threshold, causing the infusion pump to deliver an amount of a medicament to the user according to the dosing instructions.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIG. 1 is a medical device that can be used with embodiments of the disclosure, according to various embodiments of the present disclosure.

FIG. 2 is a block diagram representing a medical device that can be used with embodiments of the disclosure, according to various embodiments of the present disclosure.

FIGS. 3A-3B depict an embodiment of a pump system, according to various embodiments of the present disclosure.

FIG. 4 is a schematic representation of a system, according to various embodiments of the present disclosure.

FIG. 5 is an example simulation tree that could be created using a model-predictive-control (MPC) algorithm, according to various embodiments of the present disclosure.

FIG. 6 is a block diagram of a system for employing an automated glycemic control algorithm, according to various embodiments of the present disclosure.

FIG. 7 is a flow chart of a method for delivering individualized insulin adjustments, according to various embodiments of the present disclosure.

FIG. 8 is a visualization of the backward pass process, according to various embodiments of the present disclosure.

FIG. 9 is a visualization of the process of calculating cumulative costs for each stage, according to various embodiments of the present disclosure.

FIG. 10 is a representation of an example policy, according to various embodiments of the present disclosure.

FIG. 11 is a representation of a control law, according to various embodiments of the present disclosure.

FIG. 12 is a graph of a two-dimensional array policy that can be stored on an infusion pump, according to various embodiments of the present disclosure.

FIG. 13 is a graph of a one-dimensional array, according to various embodiments of the present disclosure.

FIG. 14 is a control diagram of an automated glycemic control algorithm with a two-dimensional policy, according to various embodiments of the present disclosure.

FIG. 15 is a control diagram of a simplified automated glycemic control algorithm with a one-dimensional policy, according to various embodiments of the present disclosure.

FIG. 16 is a graph of a unmetabolized insulin (UMI) target for 50 policies arrive, according to various embodiments of the present disclosure.

FIG. 17A is a graph of a policy, according to various embodiments of the present disclosure.

FIG. 17B is a graph of the policy of FIG. 17A with a flat portion, a quick rise portion, and a slow rise portion identified, according to various embodiments of the present disclosure.

FIG. 17C is a graph of the policy of FIG. 17A with a max flat glucose and max dose identified, according to various embodiments of the present disclosure.

FIG. 17D is a graph of the policy of FIG. 17A with indications of the impacts of adjusting automated glycemic control algorithm parameters identified, according to various embodiments of the present disclosure.

FIG. 18 is a representation of proper and improper fundamental policies with the same insulin delay characteristics, according to various embodiments of the present disclosure.

FIG. 19 is a flow chart of a method for matching policies, according to various embodiments of the present disclosure.

FIG. 20 illustrates an example system for closed- or semi-closed-loop operation of an infusion pump, according to various embodiments of the present disclosure.

FIGS. 21A and 21B illustrate optimal aIOB predictions and guardrails for the same, according to various embodiments of the present disclosure.

FIG. 22 illustrates another example system for closed- or semi-closed-loop operation of an infusion pump, according to various embodiments of the present disclosure.

FIG. 23 is a flow chart of a method for neural-network-assisted operation of an infusion pump, according to various embodiments of the present disclosure.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

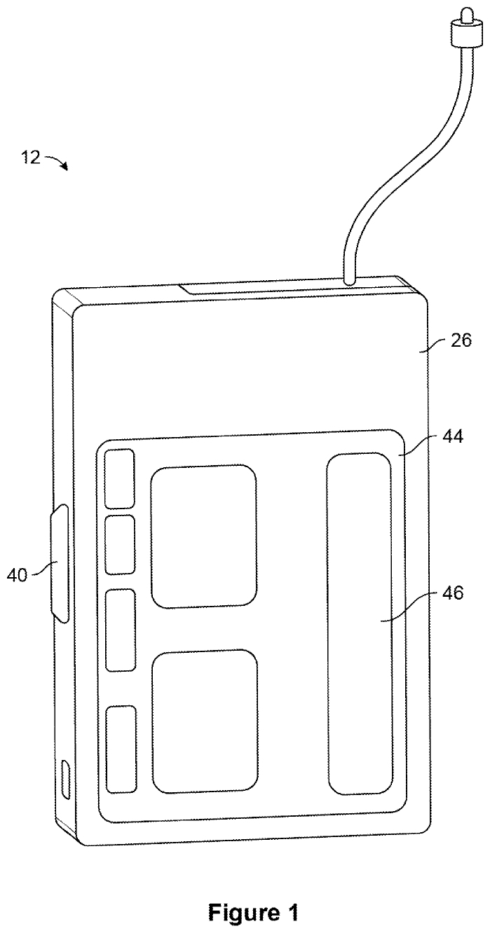

FIG. 1 depicts an embodiment of a medical device according to the disclosure. In this embodiment, the medical device is configured as a pump 12. Pump 12 may be an infusion pump that includes a pumping or delivery mechanism and reservoir for delivering medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display, a remote-control device, a laptop computer, personal computer, tablet computer, a mobile communication device such as a smartphone, a wearable electronic watch or electronic health or fitness monitor, or personal digital assistant (PDA), a CGM display etc.

In one embodiment, the medical device can be an ambulatory insulin pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

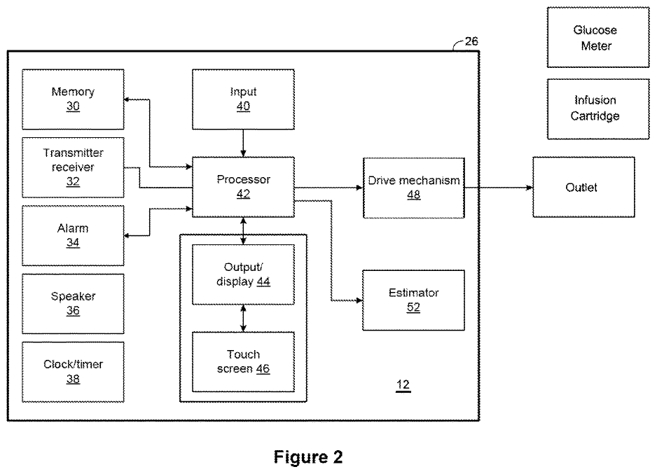

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices, for example, a CGM, display device, smartphone, etc. through the transmitter/receiver. The processor 42 may also include programming that may allow the processor to receive signals and/or other data from an input device, such as a sensor that may sense pressure, temperature, or other parameters.

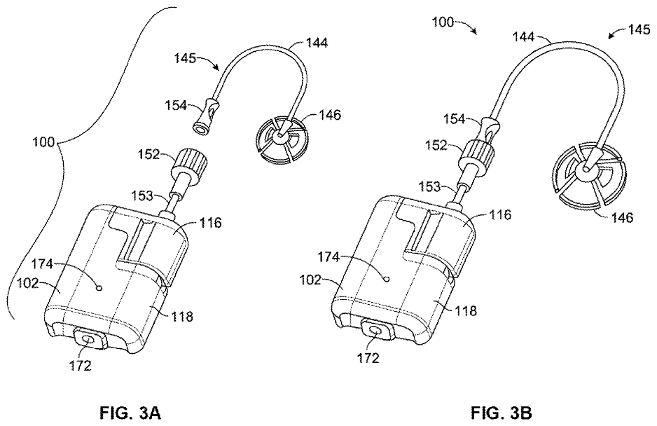

FIGS. 3A-3B depict another pump system including a pump 102 that can be used with embodiments. Drive unit 118 of pump 102 includes a drive mechanism 12 that mates with a recess in disposable cartridge 116 of pump 102 to attach the cartridge 116 to the drive unit 118. Pump system 100 can further include an infusion set 145 having a connector 154 that connects to a connector 152 attached to pump 102 with tubing 153. Tubing 144 extends to a site connector 146 that can attach or be pre-connected to a cannula and/or infusion needle that punctures the patient's skin at the infusion site to deliver medicament from the pump 102 to the patient via infusion set 145. In some embodiments, pump can include a user input button 172 and an indicator light 174 to provide feedback to the user.

In one embodiment, pump 102 includes a processor that controls operations of the pump and, in some embodiments, may receive commands from a separate device for control of operations of the pump. Such a separate device can include, for example, a dedicated remote control or a smartphone or other consumer electronic device executing an application configured to enable the device to transmit operating commands to the processor of pump 102. In some embodiments, processor can also transmit information to one or more separate devices, such as information pertaining to device parameters, alarms, reminders, pump status, etc. In one embodiment pump 102 does not include a display but may include one or more indicator lights 174 and/or one or more input buttons 172. Pump 102 can also incorporate any or all of the features described with respect to pump 12 in FIG. 2. Further details regarding such pumps can be found in U.S. Pat. No. 10,279,106 and U.S. Patent Publication Nos. 2016/0339172 and 2017/0049957, each of which is hereby incorporated herein by reference in its entirety.

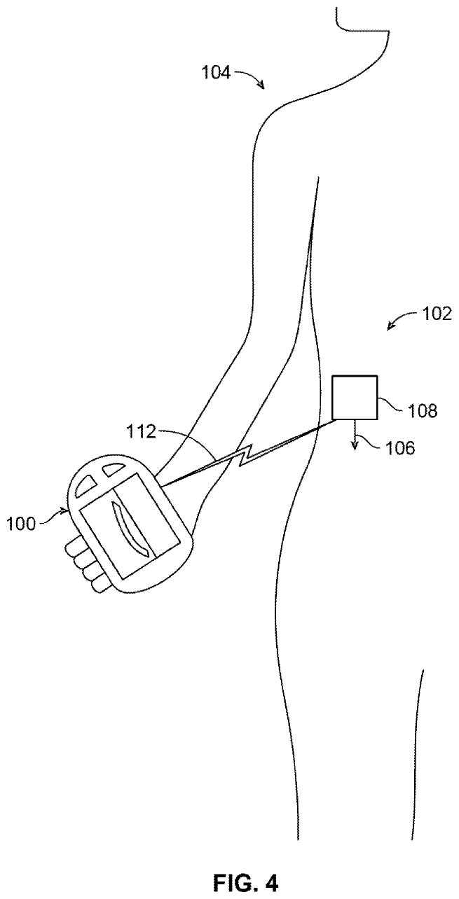

Pump 12 or 102 can interface directly or indirectly (via, e.g., a smartphone or other device) with a glucose meter, such as a blood glucose meter (BGM) or a CGM. Referring to FIG. 4, an exemplary CGM system 100 according to an embodiment of the present invention is shown (other CGM systems can be used). The illustrated CGM system includes a sensor 101 affixed to a patient 104 that can be associated with the insulin infusion device in a CGM-pump system. The sensor 101 includes a sensor probe 106 configured to be inserted to a point below the dermal layer (skin) of the patient 104. The sensor probe 106 is therefore exposed to the patient's interstitial fluid or plasma beneath the skin and reacts with that interstitial fluid to produce a signal that can be associated with the patient's blood glucose level. The sensor 101 includes a sensor body 108 that transmits data associated with the interstitial fluid to which the sensor probe 106 is exposed. The data may be transmitted from the sensor 101 to the glucose monitoring system receiver 100 via a wireless transmitter, such as a near field communication (NFC) radio frequency (RF) transmitter or a transmitter operating according to a “Wi-Fi” or Bluetooth® protocol, Bluetooth® low energy protocol or the like, or the data may be transmitted via a wire connector from the sensor 101 to the monitoring system 100. Transmission of sensor data to the glucose monitoring system receiver by wireless or wired connection is represented in FIG. 4 by the arrow line 112. Further detail regarding such systems and definitions of related terms can be found in, e.g., U.S. Pat. Nos. 8,311,749, 7,711,402 and 7,497,827, each of which is hereby incorporated by reference in its entirety.

In an embodiment of a pump-CGM system having a pump 12, 102 that communicates with a CGM and that integrates CGM data and pump data as described herein, the CGM can automatically transmit the glucose data to the pump. The pump can then automatically determine therapy parameters and deliver medicament based on the data. Such an automatic pump-CGM system for insulin delivery can be referred to as an automated insulin delivery (AID) or an artificial pancreas system that provides closed-loop therapy to the patient to approximate or even mimic the natural functions of a healthy pancreas. In such a system, insulin doses are calculated based on the CGM readings (that may or may not be automatically transmitted to the pump) and are automatically delivered to the patient at least in part based on the CGM reading(s). In various embodiments, doses can be delivered as automated correction boluses and/or automated increases or decreases to a basal rate. Insulin doses can also be administered based on current glucose levels and/or predicted future glucoses levels based on current and past glucose levels.

For example, if the CGM indicates that the user has a high blood glucose level or hyperglycemia, the system can automatically calculate an insulin dose necessary to reduce the user's blood glucose level below a threshold level or to a target level and automatically deliver the dose. If the CGM data indicates that the user has a low blood glucose level or hypoglycemia, the system can, for example, automatically reduce a basal rate and/or make other suggestions as may be appropriate to address the hypoglycemic condition. As with other parameters related to therapy, such thresholds and target values can be stored in memory located in the pump and the pump processor can periodically and/or continually execute instructions for a checking function that accesses these data in memory, compares them with data received from the CGM and acts accordingly to adjust therapy. The complexity of the algorithm used to calculate the insulin doses is therefore limited by the capabilities of the pump processor, memory, battery, etc. As such, current designs for AID infusion pump systems employ relatively simple algorithms employing only one control variable and one target and that are not personalized for individual users.

One known method for closed loop diabetes therapy is model predictive control (MPC) algorithms that have been employed by diabetes researchers using computing systems having far greater capabilities than those of a user-wearable infusion pump. The general concept behind an MPC algorithm is use of multiple control variables with multiple targets to simulate thousands (or even millions) of scenarios for each decision point. Essentially, whereas a simple algorithm will determine a single dose at a given time that will result in optimal glucose in, e.g., 30 minutes, an MPC system will determine a sequence of doses (e.g., every five minutes) that will result in optimal glucose over a much longer period (e.g., 4 hours). For example, in an MPC system each time a dose is calculated, such as every five minutes, the MPC will calculate a dose for now, a dose for five minutes from now, ten minutes from now, etc., every five minutes for the entire control horizon, such as 4 hours. If, for example, each calculation considers 100 different options, the number of simulations carried out for each dose would be 100^{(12*4)}.

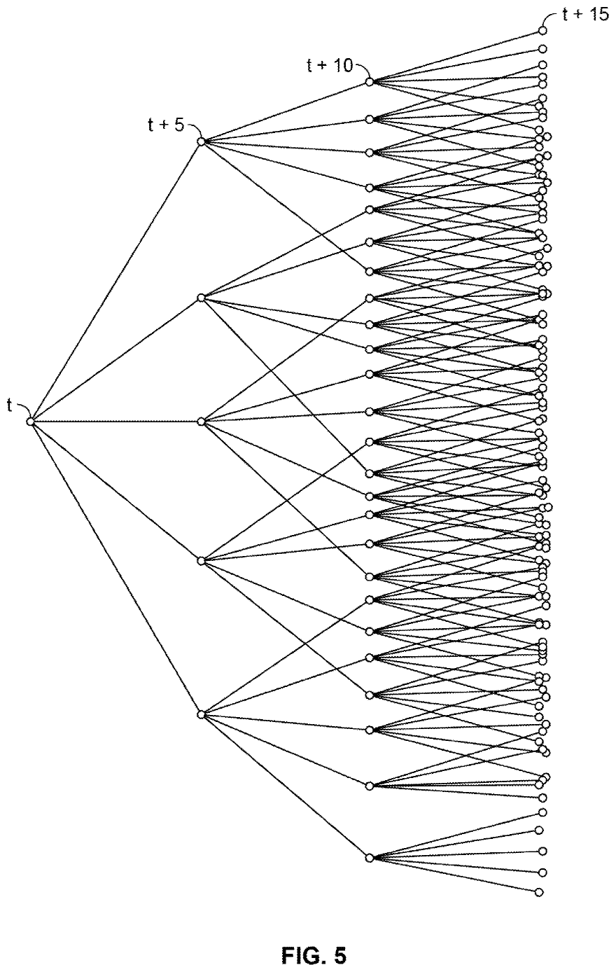

A simplified schematic representation of such a system is depicted in FIG. 5. In this simplified representation, only 5 dose options are evaluated at each decision point and only 15 minutes of simulations are depicted. For each dose evaluated at time t, the system simulates five different doses at time t+5, with five doses from each t+5 dose at time t+10, etc. The MPC selects the path that yields the best results (i.e., glucose remaining at target) and delivers the corresponding dose at time t. After the optimal dose is selected based on the simulations, the system will repeat the simulations each five minutes with updated data and with the control horizon window moving five minutes further into the future. Such a system is therefore extremely computational expensive in terms of the memory and processor resources as well as battery drain and cannot practically be carried out by small devices such as ambulatory infusion pumps.

Conventional approaches for implementing closed loop diabetes therapy in AID infusion pumps sought to develop alternatives to MPC due to the prohibitive required computational load. One such approach has been Automatic Control Systems (ACS) that implement state control optimization and rely on simplifying assumptions. These ACS include Proportional-Integral-Derivative (PID) controllers and Control-IQ (CIQ), which both incorporate state control to optimize insulin delivery to a single future point in time, rather than the event horizon contemplated by MPC. Accordingly, controllers relying on state control seek to repeatedly determine what the best blood glucose level is for a particular time (e.g., 30 minutes from now).

Whereas single state control as discussed above optimizes a single variable, such as, e.g. a glucose level 30 minutes in the future (i.e., gPred30), MPC enables an objective function that can consider a plurality of items, such as, for example, current or predicted future glucose, time below a low threshold (e.g., 70 mg/dL, 54, mg/dL, etc.) insulin dosage, glucose velocity etc.

Further, as with any ACS, both PID and CIQ rely on simplifying assumptions. The fundamental assumptions of PID are that the system being controlled is simple, symmetric, and linear; however, PID may operate even when one or more of these assumptions are violated. This enables PID controllers to only consider the error signal across the whole range. CIQ fundamentally assumes that the system being controlled is linear but not simple or symmetric, enabling basal insulin increase control to be accomplished by considering only the error signal when blood glucose is above a threshold. In both cases, these conventional approaches assume linearity and base dosing on error. However, it is well understood that the human body is nonlinear. For example, a unit of insulin acting on a person who is at 200 mg/dL will not cause the same (or even proportionate) response to a person who is at 70 mg/dL. Thus, conventionally contemplated methods of closed loop diabetes therapy in AID infusion pumps face significant obstacles based on the assumptions and shortcuts made to simplify computational overhead.

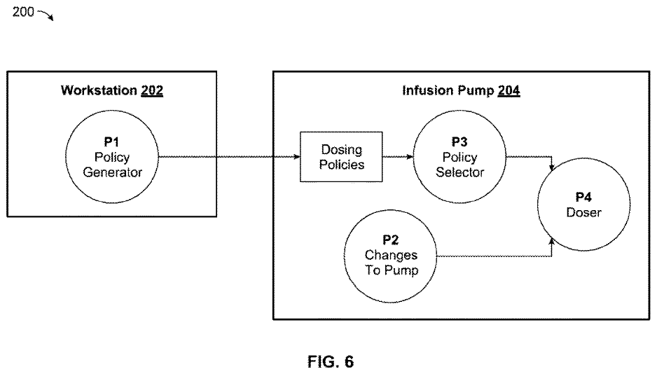

Embodiments disclosed herein employ a dynamic and interoperable automated glycemic control algorithm that conforms to the technical definition of an MPC algorithm while employing dynamic programming to reduce efficiency concerns associated with such algorithms. Referring to FIG. 6, a block diagram of a system 200 for employing the automated glycemic control algorithm of the present disclosure is depicted according to embodiments. System 200 implements the automated glycemic control algorithm across a workstation 202 and an infusion pump 204 to provide optimized insulin delivery based on four processes (P1, P2, P3, and P4).

At P1, a dosing function (or dosing policy) generation process is performed at workstation 202. The policy generation process incorporates the combination of MPC and dynamic programming. 131 reduces the control law for the infusion pump into lookup tables or “policies” that will be described in greater detail later on. The P1 logic resides on workstation 202 and can generate the policies offline. These policies can then be loaded onto the pump during the manufacturing process or through a programmer. In embodiments, P1 can be implemented on any computing devices capable of remotely or locally handling the associated computational overhead, such as a smartphone, a tablet computer, a laptop computer, a desktop computer, or a high-end workstation.

At P2, required changes to the infusion pump to accommodate the policy are determined. While not technically part of the policy generation and selection process, P2 includes the calculation of any modifications necessary to facilitate dosing using the automated glycemic control algorithm.

At P3, the infusion pump logic considers a user's characteristics to (a) select the most appropriate policy and (b) scale/stretch the policy per the user's current settings. To match users with dosing functions, the automated glycemic control algorithm considers four inputs: target blood glucose, basal, correction factor, and carb ratio. These four values can be manually entered by a user or automatically derived from the user's open loop profiles.

At P4, the automated glycemic control algorithm uses the selected policy, the dosing history, and CGM data to select the optimum dose for each five-minute period. This P4 logic includes modules for estimating UMI, approximating insulin onboard (IOB), and generating glucose predictions. Importantly, P4 considers all insulin active in the body instead of merely deviations from a basal rate (IOB). Estimating UMI at a certain time (t) can be governed by the following:

$$U ( t ) = {U}_{o} {e}^{- kt} . \qquad Equation 1$$

$$UMI Estimation for a Dose at Time t .$$

Because this UMI estimation is linear, the burn downs from multiple doses are expected to combine linearly. Accordingly, the solution for a dose at t=5 and another dose at t=15 may be found by summing the two burn downs.

The burn down for each time segment can be estimated by (i) adding in the new dose and (ii) multiplying the sum by a constant. Equation 1 can be used to determine the ratio of subsequent doses which simplifies to:

$$M = {e}^{- 5 k} . \qquad Equation 2$$

$$Ratio of Subsequent Doses .$$

Here, k is the UMI burn down constant. Because k is a constant, M is a constant and it can be concluded that the ratio for subsequent solutions is constant. This means subsequent solutions may be found simply by multiplying by M.

When dosed per a single basal rate with no boluses, the value of UMI will settle to the value basal rate divided by k. The value is used to initialize the UMI estimates:

$$U ( ∞ ) = \frac{B}{k} . \qquad Equation 3$$

$$UMI for Basal Only .$$

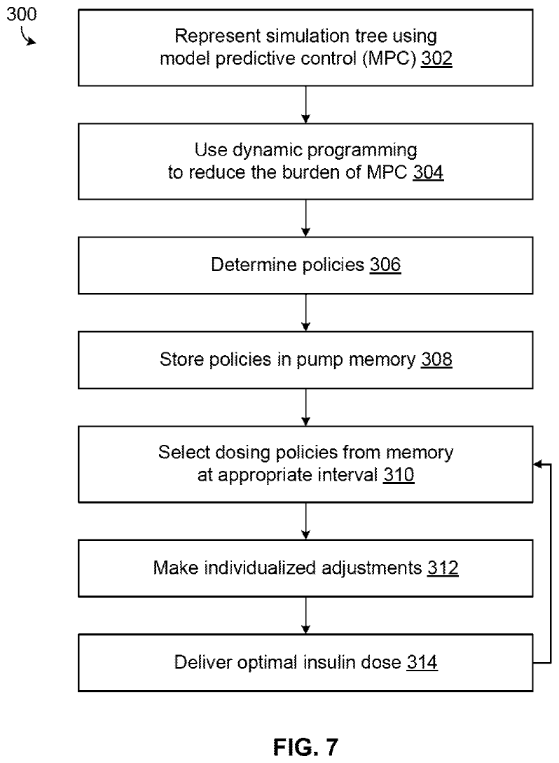

Contemplated herein is the employment of dynamic programming to perform offline computations, i.e., not done on the pump, to create a series of dosing lookup tables each of which can be considered a policy. These policies simplify the computational burden placed on the insulin pump to look-up operations. Referring now to FIG. 7, a flow chart of a method 300 for delivering individualized insulin adjustments based on such policies is depicted according to an embodiment. In embodiments, method 300 can be implemented through system 200.

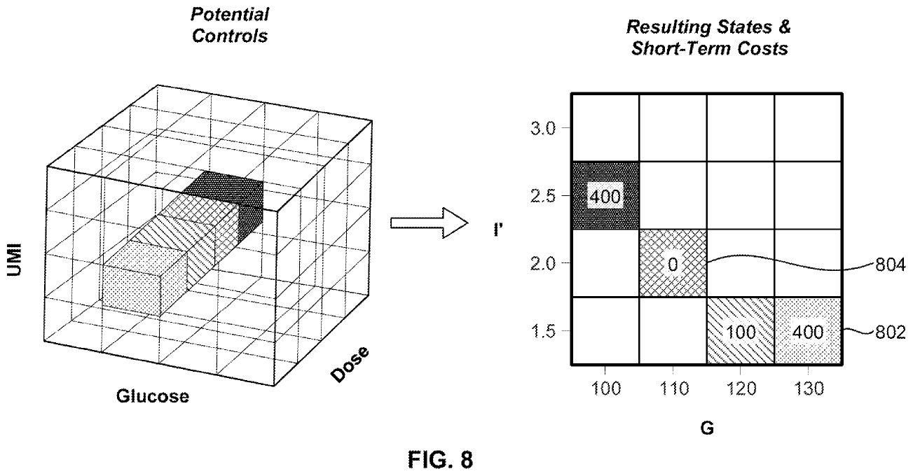

It is contemplated that the processing logic implemented from step 302 to step 306 can be ran on a workstation independent from an infusion pump. The computations can be performed on a computing system having computational capabilities greater than those of an ambulatory infusion pump. At step 302, a simulation tree (e.g., the simulation tree depicted in FIG. 5) is represented using MPC, where the representation may implicitly contain the same possible paths as the simulation tree and may be in a tabular form such as a look-up table, which is initially empty. At step 304, dynamic programming is used to reduce the burden of MPC by removing unnecessary calculations as will be described with regards to FIGS. 8-11. Step 304 works backward on the implicit representation of the simulation tree in the table and fills out the empty table which becomes the policies/lookup tables. At step 306, the policies, or lookup tables, are determined based on a backward pass of the simulation tree.

These policies can then be stored in the memory of the pump and/or remote control at step 308. At step 310 embedded logic in the pump and/or remote control can select dosing functions from the memory to determine appropriate doses for each dosing interval (i.e., every five minutes). This implementation effectively simplifies real-time computation to a lookup operation by the infusion pump. At step 312, individualized adjustments can be made based on one or more of (i) a target blood glucose, (ii) basal, (iii) correction factor, and (iv) carb ratio. Finally, at step 314, the insulin pump delivers the determined insulin dose. Fundamentally, the automated glycemic control algorithm works by regulating UMI such that UMITarget is elevated at high predicted glucose values and reduced at low predicted glucose values. The error term is defined as:

$${UMI}_{Residual} = {UMI}_{Target} - {UMI}_{Estimate} . \qquad Equation 4$$

$$Error Term for Dose Computation .$$

Steps 310 through 314 can be repeated as necessary to enable the automated glycemic control algorithm to operate ad infinitum without running out of policies.

It should be understood that the individual operations used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

### Using Dynamic Programming to Create an MP for Insulin Dosing

As aforementioned, embodiments disclosed herein employ dynamic programming in order to utilize a MPC system with an ambulatory infusion pump. Application of dynamic programming to an MPC system for diabetes therapy simplifies the calculations by (i) traversing the simulation tree such as that depicted in FIG. 5 in reverse and (ii) preventing duplicative calculations by memorizing all previous results (i.e., storing results and recalling the previous calculation when the same inputs occur again rather than re-calculating). Through this novel incorporation of dynamic programming to reduce the efficiency concerns normally associated with MPC algorithms the inventors have discovered that regardless of boundary conditions, dosing patterns emerge. These dosing patterns are the policies. There are five requirements to produce policies via dynamic programming. These are (i) a predictive model of glucose and insulin, (ii) a cost function, (iii) a terminal state, (iv) a backward pass, and (v) a means of storing the policies.

### Predictive Models

The predictive model is the underlying core of MPC and allows prediction of the effects of any dosing decision. In embodiments, simplified models can be represented as follows:

$$\frac{dG}{d t} = D - ( {m}_{g} + {I}^{′} ) G . \qquad Equation 5$$

$$Glucose Subsystem .$$

$$\frac{{dI}^{′}}{d t} = {B}^{′} - {m}_{i} {I}^{′} . \qquad Equation 6$$

$$Insulin Subsystem .$$

wherein G is representative of glucose (e.g., in mg/dL), D is representative of endogenous glucose production (e.g., in mg/dL/min), m_{g} is representative of glucose mass action constant (e.g., in I/min), I′ is representative of unmetabolized insulin (“UMI”) (e.g., in units), B′ is representative of dose rate (e.g., in units/min), mᵢ is representative of insulin decay constant (e.g., 1/min).

The two variables G and I′ describe the metabolic state and any point in time and are referred to as “state variables.” The constants D, m_{g}, and mᵢ describe how an individual's metabolism will behave over time and are referred to as “metabolic parameters.” The value B′ describes how much insulin is administered at each point in time. Therefore, B′ is the quantity that can be controlled in order to optimize a user's glucose levels. As such, B′ is referred to as the “control variable” or simply the “control.”

Such predictive models allow present states and controls to be mapped to future states. For example, the predictive model can be used to simulate a hypothetical state transition after dosing 1 unit of insulin over a single time frame.

### Cost Function

The cost function serves as a way of evaluating future states to determine which control is optimal. Accordingly, the cost function is an equation that calculates the relative “cost” or “undesirableness” of a state. In embodiments, the cost function can be represented as follows:

$$Cost ( G ( t ) ) = {\int }_{{t}_{o}}^{{t}_{f}} {( H ( t ) ( G ( t ) - {G}_{target} ) )}^{2} dt . \qquad Equation 7$$

$$Cost Function for automated glycemic control algorithm .$$

$$H ( t ) = \{ \begin{matrix}1 if G ( t ) > {G}_{target} \\ β otherwise , where β > 1\end{matrix} . \qquad Equation 8$$

$$Hypoglycemia Bias Term , or “ Hypo Penalty . ”$$

By squaring the difference between G(t) and G_{target}, glucose values that are farther away from G_{target} are penalized more than proportionally than values that are close to G_{target}. Further, squaring the difference ensures that the cost is always positive. Applying the hypo penalty H(t) to values that fall below G_{target} enables the policy derivation process to be biased so that it is more averse to hypoglycemia.

In embodiments, this cost function can be used to determine both the short-term and long-term costs of each control during the backward pass to evaluate every possible sequence of controls.

### Terminal State

The terminal state is the starting point for the backward pass, which is an iterative process. The terminal state represents the outcome of following a sequence of dosing decisions over a finite amount of time known as a “finite time horizon.” Rather than trying to simulate all the possible dosing sequences and then determine their resulting terminal states (which would be computationally infeasible), it is contemplated that one could observe a finite number of states in which an individual's metabolism could terminate. Upon reaching each of these states, a user will no longer receive closed loop control but will instead receive only their open loop basal rate going forward.

Each of the possible terminal states can be used as a starting point to simulate the user's metabolism under open loop basal rate over a terminal horizon. In embodiments, the terminal horizon can be six-hours. Notably, the terminal horizon is distinct from a time horizon over which the backward pass occurs. Instead, each terminal horizon enables a determination of the long-term consequences of a user ending up at the associated terminal state. The cost function is applied to each glucose trace produced by the open loop simulations (e.g. time horizons) to arrive at a terminal cost for each terminal state. These terminal costs can therefore represent the desirability of each terminal state.

In operation, the terminal cost of ending up at G_{target} and I_{equilibrium} will be essentially zero or lower than other termination states. This is because that state represents the stable equilibrium at which a constant basal rate will cause glucose to remain at exactly G_{target}. Reaching a terminal state with too much unmetabolized insulin will result in a glucose trace that falls below G_{target} over the next six hours and produces a high cost. On the other hand, reaching a terminal state with too little unmetabolized insulin will cause glucose to rise above G_{target} before falling back down, resulting in a moderate cost.

### Backward Pass

Once the terminal costs are computed, a backward pass can be taken from the terminal states to determine the optimal control for each state at each point in time. The backward pass is a key element of implementing the dynamic program. The backward pass incorporates analysis after time segments known as “stages.” In embodiments, each stage can be five minutes (but may be of shorter or longer duration). At each stage, the short-term and long-term costs of all the possible controls (dose decisions) to be made in all the states is considered. The short-term cost of each control can be determined by simulating a stage and applying the cost function to the resulting glucose-insulin state. The long-term cost of each control can be similarly determined by referencing the previously calculated cumulative cost of the resulting glucose-insulin state. As the backward pass is conducted, the short-term and long-term costs are stored, recorded, or “memo-ized” (i.e. make a memo of) so that these values can be used in following iterations.

For a given state of unmetabolized insulin (or all insulin onboard), I′, and glucose. G, there are many possible dose values or control values that can be selected. The states resulting from each of these controls are analyzed to determine cumulative costs. Cumulative costs are determined by adding short-term costs with the long-term costs that were memo-ized in the previous iteration. This process enables selection of the control that produces the optimal cumulative cost, accounting for both short-term and long-term consequences. This process is repeated for every glucose-insulin state at each stage, resulting in a full matrix of optimal controls, known as a “dose policy” (which is interchangeably referred to as “policy” or “dosing function” in this disclosure).

FIGS. 8-10 depict an example of the process for creating a policy. A partial visualization of the backward pass process is shown in FIG. 8 by a three-dimensional matrix of I′, G, and B. For a given state of I′ and G there are many possible dose or control values that can be selected as represented by each block within the matrix. The states resulting from each of these controls are shown in the panel on the right. In this example, the smallest control, represented by block 802, results in a higher glucose state and lower insulin state. This indicates that unmetabolized insulin is decaying and the metabolism is producing glucose endogenously. To maintain equilibrium, a larger control is necessary. In this case, the optimal control is represented by block 804, which has the lowest calculated cost of zero.

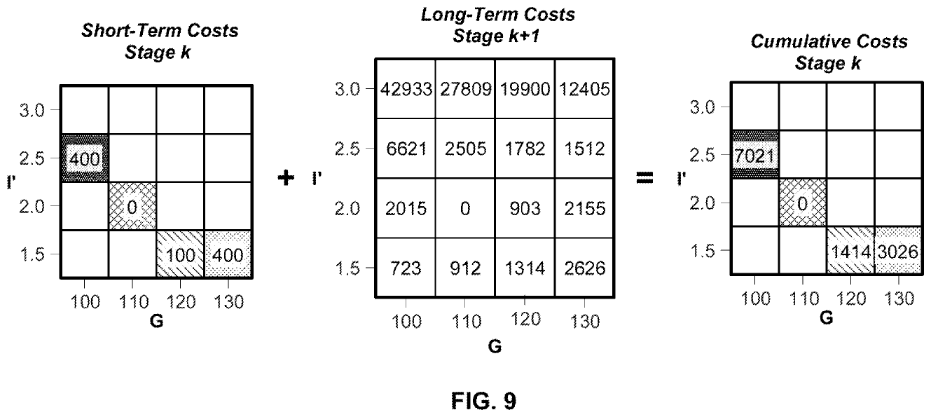

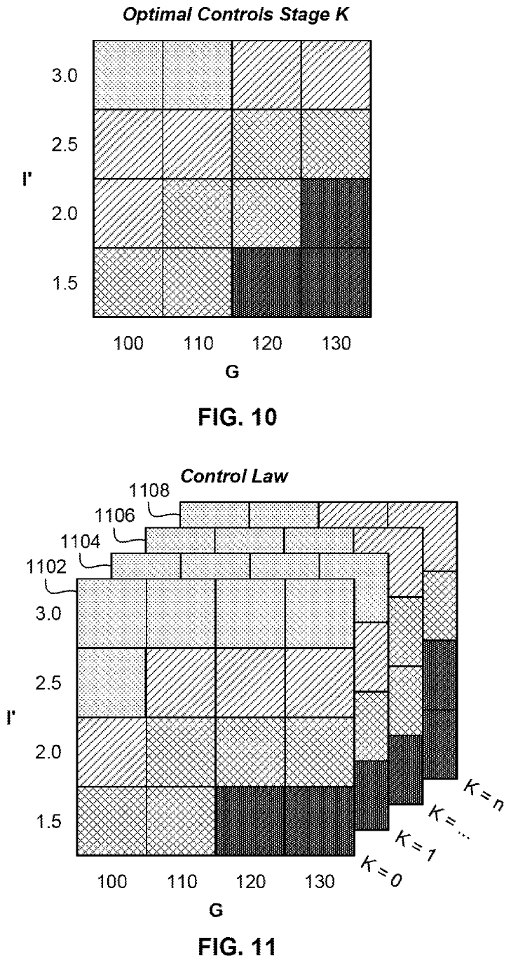

FIG. 9 depicts the process of calculating cumulative costs for each stage. As shown, cumulative costs are determined by adding short-term costs with the long-term costs that were calculated in the previous iteration. In embodiments, these long-term costs are memoized to reduce computational overhead, as previously described. Although only partially represented in FIG. 9, cumulative cost calculations can be repeated for every glucose-insulin state at stage k, resulting in a full matrix of optimal controls known as a policy (also referred to herein as a dosing function or a dosing policy). A representation of an example policy is depicted in FIG. 10.

Thus, at every stage in time, the backward step process can dynamically select the optimal control. That control and the corresponding policy can be used to determine the best way to dose insulin given a user's current glucose-insulin state. Across stages these policies build upon each other resulting in a sequence of optimal control policies known as a “control law.” A representation of a control law is depicted in FIG. 11, which includes an optimal policy for K equals 0 (policy 1102), an optimal policy for K equals 1 (policy 1104), an optimal policy for some intermediary value of K (policy 1106), and an optimal policy for K equals some n (policy 1108). For example, the first policy 1102 may indicate that, for example, (i) when glucose is between 100 and 110, that optimal control occurs at a UMI of 1.5, or (ii) when glucose is between 110 and 130, that optimal control occurs at a UMI of 2.

For example, an embodiment incorporating a finite time horizon of six hours with a stage of five minutes would result in an optimal closed-loop control law being calculated based on 72 stages. This six-hour horizon would then terminate with the user receiving open loop basal rate for an additional six hours thereafter. In total, 12 hours of control would be simulated. While 72 closed-loop control policies would be derived for this example, only the policy 1102 derived for stage K equals 0 would be used in making dosing decisions on the pump. This causes the on-pump bolus rate controller/recommender (e.g., AIDA (Adapting Insulin Delivery to Activity)) to always select the control that optimizes the next six hours of closed-loop dosing, creating a receding time horizon. Essentially, the backward pass process allows the AIDA to operate ad infinitum without running out of policies. In embodiments, these policies and resulting control laws can all be calculated at a workstation external to an infusion pump. However, in some embodiments, the policies and resulting control laws are calculated using a processing device and/or processing software implemented within an infusion pump.

Optimal control laws can be derived for different metabolisms by representing each metabolism by a unique combination of the metabolic parameters D, m_i, and m_g. Metabolisms can be chosen due to the acceptable performance of their corresponding zeroth stage control policies in controlling simulated pump users. In some embodiments, for each metabolism, only the stage K equals 0 policy 1102 is uploaded to the pump. In such embodiments, the total number of policies uploaded to the pump is one for each unique metabolism. In an embodiment, one policy for each of 15 metabolisms can be uploaded to the pump.

### Means of Storing Policies

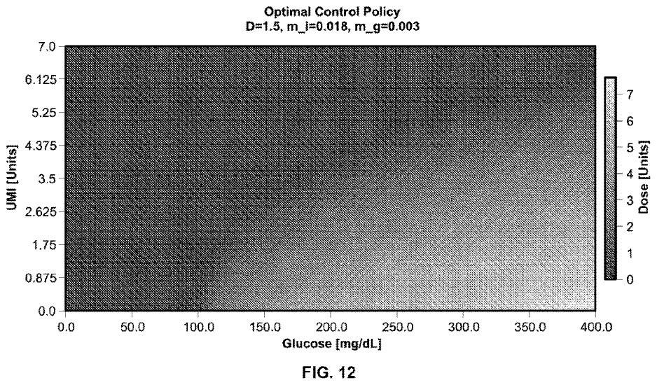

The policies can be uploaded onto the pump in a variety of ways, each with particular benefits. In one embodiment, the method of upload involves uploading the entire two-dimensional, stage K equals 0 policy 1102. An example of such a two-dimensional policy is shown in FIG. 12 as a 152×81 array, with optimal controls stored in each cell and represented visually by the color scale. This example policy indicates that if a user's CGM reading is 200 mg/dL and their computed UMI is approximately 2 units, the optimal dose is 2.7 units.

Storing policies on-pump as two-dimensional arrays (or lookup tables) is beneficial because this method is intuitive and requires minimal computation from the pump's processor in order to make dosing decisions in real time. The pump's processor only needs to look up the optimal dose in this two-dimensional table based on the CGM reading and the UMI, which the pump keeps track of as it administers insulin overtime. For example, the array shown in FIG. 12 can take up just 98.5 kilobytes of memory and a total footprint for 15 of these policies would be less than two megabytes.

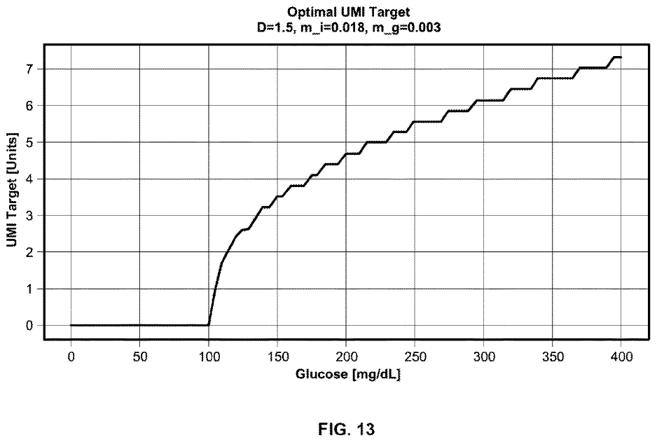

A second approach for storing policies is based on a novel pattern recognized by the inventors of the present disclosure—that for any given glucose state the prescribed dose varies inversely with insulin but the resulting insulin state remains constant. In other words, while the doses may vary depending on how much insulin is currently in the user's body, the new insulin state, or “UMI target,” does not. Accordingly, it has been observed that the UMI target only varies with glucose. As such, the policies can be compressed by storing a one-dimensional UMI target array on the pump instead of the two-dimensional dose lookup table. FIG. 13 depicts an example one-dimensional array with 81 values. Each value represents the UMI target for the corresponding glucose state. The size of this example one-dimensional array is just 648 bytes; the total footprint for 15 of these UMI target arrays would be less than 10 kilobytes.

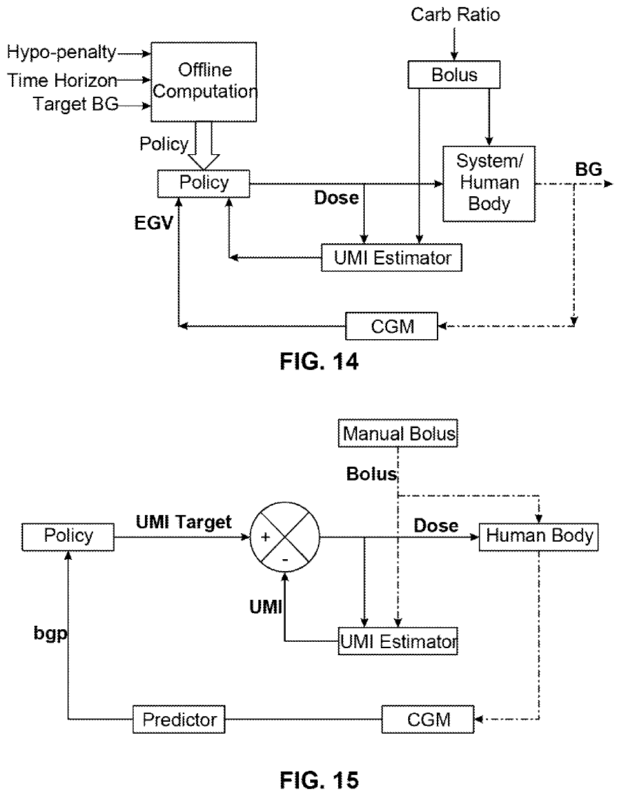

The novel realization that the sum of active insulin and dose is constant for each policy enabled use of one-dimensional policies and further compressed storage of the policy lookup table. The benefits of this realization can be seen in part by contrasting FIG. 14, depicting an automated glycemic control algorithm control diagram with a two-dimensional policy, with FIG. 15, a simplified automated glycemic control algorithm control diagram with a one-dimensional policy.

A third means of storing policies on the pump involves fitting a function to the UMI target curve. Success has been found in fitting UMI target curves to a cube-root function of the form:

$${UMI}_{Target} = a + {b}^{3} \sqrt{( Glucose + c )} . \qquad Equation 9$$

$$Cube - Root Function for UMI Target Curves .$$

Fitting the UMI target curves in this manner would enable representation of the entire dosing function with only three fitted constants: a, b, and c. Using such an approach, the total footprint for 15 policies would be less than 400 bytes.

One caveat to consider with the second and third storage methodologies for storing policies is that, while less space in memory is used, more computation from the pump's processor is required in order to translate the values into dose rate commands. Accordingly, these methods require the pump's processor to compare current UMI with the looked-up UMI target and then compute the necessary dose rate to achieve that target. In contrast, the first method of storing a two-dimensional array stores the actual dose rates for lookup, reducing computational overhead.

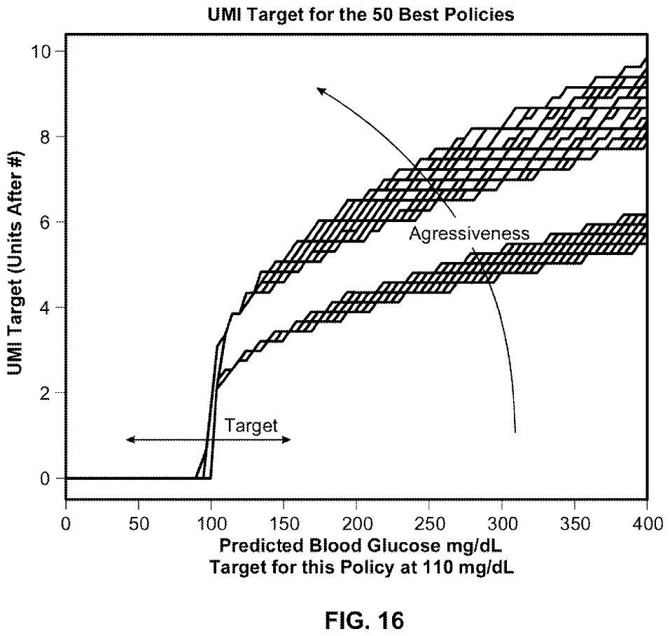

Regardless of the storage method chosen, the front loading of the computational overhead, such as via an offline workstation, enables only the policies to be stored on the pump. Additionally, the minimal file sizes of policies allow for many different control laws, each for unique metabolisms, to be stored within the infusion pump. FIG. 16 depicts a how 50 policies can each arrive at a UMI target of 110 mg/dL.

### Additional Considerations

Other considerations of the present disclosure should be considered in addition to the five aforementioned elements. One such consideration is that insulin and glucose states are continuous and can be any value within a range. For the sake of computational feasibility, these quantities must be discretized. In some embodiments, glucose can be represented as a multiple of, e.g., 1 or 5 from 0 to 400. Glucose values of 300.2 or 154, for example, are rounded down to the nearest multiple of 5. When graphed this rounding approach results in incremental steps as seen in FIG. 13.

Another consideration is that simulating five minutes forward from any glucose-insulin state often results in a new glucose-insulin state with values that fall between the discretized values used in the computation. Although a glucose state of 110 may be used to start, the next glucose state may be 103 or 105.7. To speed up the computation of short-term costs for these resulting states, a combination of pre-caching and bilinear interpolation can be used.

Additionally, the mapping from present states and controls to future states is time independent. For a given metabolism, dosing 1.2 [units] of insulin while in the state G=180 [mg/dL] and UMI=3.8 [units] will always result a new state of Gnew=176.42 [mg/dL] and UMInew=4.89 [units] five minutes later, regardless of stage. This enables greatly sped up computation time by pre-caching a present-to-future map and then looking up the answers rather than repeating our metabolic simulations at every stage.

Another consideration is that different users have different levels of sensitivity to insulin. While a limited number of policies are stored on the pump in some embodiments, those policies can be scaled up or down by applying a scaling factor SGI, which represents insulin sensitivity. This enables tailoring the relative dose sizes of each policy to the needs of each individual user. The value of SGI is currently found by dividing the automated glycemic control algorithm metabolic basal rate by the user's real life basal rate.

### Policy Elements and Personalization

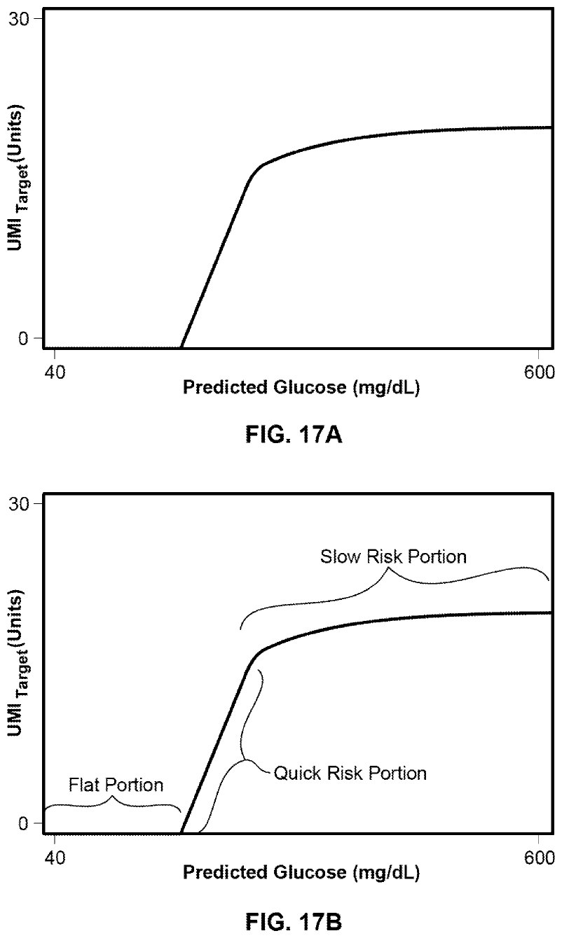

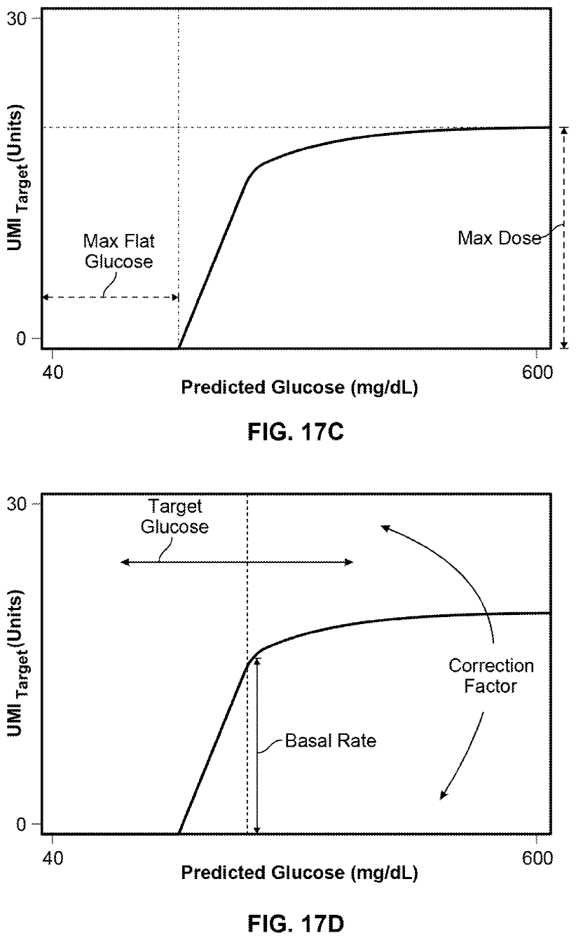

As previously described, policies are lookup tables generated by the automated glycemic control algorithm policy generator. A typical policy is depicted in FIG. 17A according to an embodiment and elements of that policy are identified in FIGS. 17B-D.

Each policy defines the relationship between the predicted glucose and the UMITarget over a range. In the embodiment depicted in FIGS. 17A-D, the policy defines the range of 40 mg/dL to 600 mg/dL.

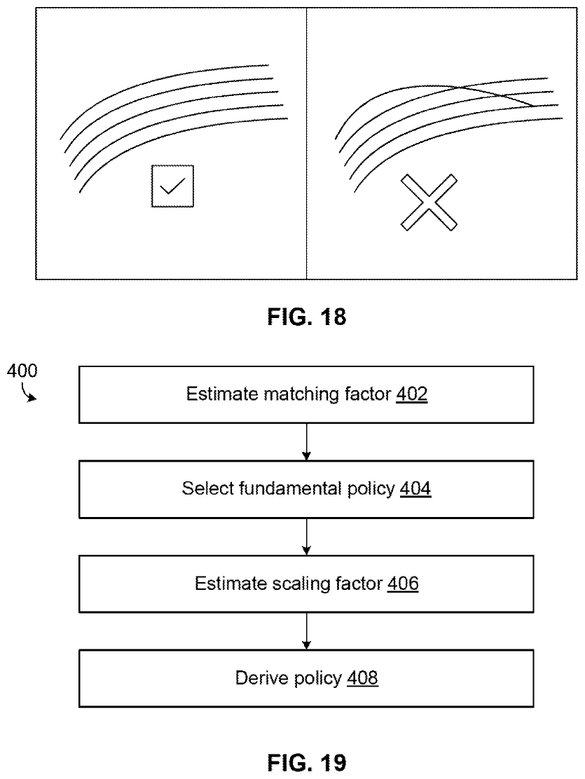

In some embodiments, the automated glycemic control algorithm can provide a minimum of 25 fundamental policies. Fundamental policies are the policies stored in Non-Volatile Memory (NVM). Fundamental policies may be mathematically expanded into user policies (or derived policies) using automated glycemic control algorithm Basal, automated glycemic control algorithm Target Glucose, and automated glycemic control algorithm Correction Factor. All user policies created from a single fundamental policy belong to the same policy family. The fundamental policies stored in NVM shall be derived such that when fundamental policies with the same insulin delay characteristics are plotted on the same graph, no fundamental policy intersects another as represented in FIG. 18.

In embodiments, alternate therapy modes can accordingly be based on different fundamental policies. Alternate therapy modes can include one or more of temporary policy, meal policy, exercise policy, sleep policy, gestational policy, and hospital policy.

In embodiments, each user policy metadata can include one or more of automated glycemic control algorithm Target Glucose, automated glycemic control algorithm Basal, automated glycemic control algorithm Correction Factor, D (Endogenous Glucose Production), SGI (Sensitivity), Max Flat Glucose, Max Dose, and Checksum.

In embodiments, each fundamental policy metadata can include one or more of D (Endogenous Glucose Production), Max Flat Glucose, Max Dose, and Checksum.

Referring to FIG. 17B, each policy consists of three segments: a flat portion, a quick rise portion, and a slow rise portion. In the flat portion, UMITarget should be zero as this represents flat glucose. In the quick rise portion, UMITarget should rise approximately linearly (sometimes following a sigmoid) with predicted glucose, and the largest y-value should occur at the predicted glucose target. In the slow rise portion, UMITarget shall have a positive first derivative and a negative second derivative.

Each policy has both (i) a maximum dose and (ii) end of flat glucose value, as depicted in FIG. 17C.

The impact of adjusting automated glycemic control algorithm parameters is illustrated in FIG. 17D. Adjusting automated glycemic control algorithm Target Glucose tends to translate the policy to the right or left. Adjusting automated glycemic control algorithm Basal tends to scale the policy up and down. Adjusting automated glycemic control algorithm Correction Factor tends to rotate the policy Clockwise (CW) or Counter-Clockwise (CCW).

Ordinarily, only one automated glycemic control algorithm user policy is active at a time. However, when fading from one policy to another, two automated glycemic control algorithm user policies may be active. In some embodiments, the automated glycemic control algorithm software checks all three integrity features of all active user policies at least once per stage.

Fundamental policies define unique shapes which may be stretched and scaled for individual users. This stretching/scaling process allows the user to make small adjustments similar to changing basal rates on other pumps. By matching users with appropriate user policies, the automated glycemic control algorithm produces previously unattainable time-in-range results, especially after meals.

Because each policy can be personalized, each user will have an optimum policy. In some embodiments, the infusion pump can be controlled using a graphical user interface (GUI) that is configured to receive user inputs and provide user outputs regarding configuration of the infusion pump and status automated glycemic control algorithm parameters. The GUI can be presented on a screen of the infusion pump or can comprise a mobile application, webbased application, or any other executable application framework. In embodiments, the GUI can reside or be presented on a smartphone, a tablet computer, laptop computer, or desktop computer.

Referring now to FIG. 19, a flowchart of method 400 for matching policies with a user is depicted according to embodiments.

It is contemplated that the processing logic implemented from step 402 to step 408 can be ran on an ambulatory infusion pump. At step 402, a matching factor is estimated by multiplying the basal rate by the correction factor. At step 404, a policy is selected that best matches the matching factor and target glucose. At step 406, the scaling factor is estimated by dividing the user's basal rate by the fundamental policy basal rate. At step 408 the derived policy is generated by multiplying the selected fundamental property by the scaling factor. The patient specific derived policy can then be applied.

In some embodiments, type settings can be presented to a user that allows for personalized medicine. Instead of entering parameters such as basal rate, insulin action time, body weight, total insulin, and target insulin, a user can simply select a policy and adjust the policy as necessary by adjusting automated glycemic control algorithm parameters through the GUI, lessening the user burden.

In embodiments, because one policy can be used for the entire range of predicted glucose, the contemplated approach is reactive and always considers both predicted glucose and total insulin in the body. These constant considerations can effectively place brakes on excess insulin—an advantage over conventional solutions such as CIP.

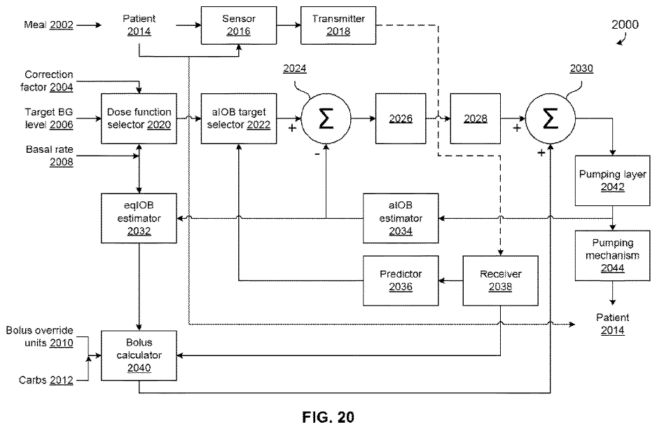

FIG. 20 illustrates an example system 2000 for closed- or semi-closed-loop operation of an infusion pump (incl., e.g., pumping layer 2042 and pumping mechanism 2044), according to various embodiments of the present disclosure. The system 2000 may also be referred to herein as an artificial pancreas system 2000, or a dynamic artificial pancreas (“DAP”) system 2000.

In the illustrated embodiment, the DAP system 2000 receives dietary, physiological, and other data regarding a patient 2014 (e.g., a person with diabetes (PWD)). For example, the data may regard a meal 2002 or carbohydrates 2012 consumed by the patient. It may include a correction factor 2004 that indicates the effectiveness of insulin on the patient 2014. Additionally, it may include a target blood glucose level 2006, a basal rate 2008, or bolus override units 2010.

The example system 2000 includes various components, devices, and/or modules that together interface and interact to ultimately determine whether and how much insulin (or other medicament) should be provided to the patient 2014. The following discussion assumes that the system includes three separate devices: (i) a CGM with a sensor 2016 (e.g., a blood glucose sensor) and a transmitter 2018 (e.g., a Bluetooth transmitter, an NFC transmitter, a Wi-Fi antenna); (ii) an electronic device with a dose function selector 2020, an all-JOB (“aIOB” or sometimes referred to as “AOB”) target selector 2022, an equivalent IOB (“eqIOB”) estimator 2032, an aIOB estimator 2034, a predictor 2036, a receiver 2038 (e.g., a Bluetooth receiver, an NFC receiver, a Wi-Fi antenna), and a bolus calculator 2040, and (iii) an infusion pump (e.g., an ambulatory infusion pump) with a pumping layer 2042 and a pumping mechanism 2044. Nonetheless, the system 2000 need not include each of these three devices, or it may include these devices but with different subcomponents and/or the subcomponents (e.g., eqIOB estimator 2032, receiver 2038, pumping layer 2042, sensor 2016) distributed differently among the devices.

In an example embodiment, after the patient 2014 eats a meal 2002, the CGM (incl. sensor 2016, and transmitter 2018) determines a blood glucose level of the patient 2014 (e.g., using the sensor 2016), and transmits data regarding the patient 2014 (e.g., using the transmitter 2018) to the receiver 2038 of the electronic device. Determining the blood glucose level of the patient 2014 may be based in part on sensor data received from the sensor 2016 of the CGM.

Additionally, in the example embodiment, the electronic device (incl. the dose function selector 2020, the aIOB target selector 2022, the eqIOB estimator 2032, the aIOB estimator 2034, the predictor 2036, the receiver 2038, and the bolus calculator 2040) receives the patient data from the CGM via the receiver 2038. The electronic device also receives other data, for example, from a user input, from another electronic device (e.g., a server), or from the infusion pump (e.g., an ambulatory infusion pump).

The computational resources required to operate the electronic device may be exceptionally small. For example, in some embodiments, the electronic device includes only 48 KB of RAM for performing the instructions discussed herein with respect to said electronic device. This may allow the electronic device to run on a CGM, a portable device connected to a CGM, a smartphone, a smartwatch, or another conveniently portable device. In this manner, the innovations discussed herein may enable particularly convenient dosing approximation all without significantly sacrificing accuracy, patient safety, or user experience.

The additional data may include the aforenoted correction factor 2004, target blood glucose level 2006, basal rate 2008, bolus override units 2010, or carbohydrates 2012 consumed by the patient 2014. The correction factor 2004 may indicate the effectiveness of insulin on the patient 2014. For example, the correction factor 2004 can be used as a metric for translating an amount of insulin into an effect on the blood glucose level of the patient. The bolus override units 2010 may indicate an amount of insulin provided manually by the patient 2014 and not automatically by the infusion pump.

Some of the data, such as the correction factor 2004, the target blood glucose level 2006, and the basal rate 2008, may be received at the dose function selector 2020. The dose function selector 2020 may then use this data to select a dosing function (or dosing policy), for example, from a plurality of dosing functions that each correspond to a particular function for administering a dose of insulin to the patient 2014 via the infusion pump (e.g., as discussed in detail above with respect to FIGS. 8-11).

After selecting the dosing function, the dose function selector 2020 may then provide the selected dosing function to the aIOB target selector 2022. From there, the aIOB target selector 2022 can then determine an aIOB target based on the selected dosing function and predicted glucose value(s) from the predictor 2036. As used herein, “all insulin on board” or “aIOB” refers to a target amount of all insulin in the patient 2014. The present disclosure also uses “unmetabolized insulin” or “UMI” to refer to this target amount of all insulin in the patient. This differs from IOB, which refers only to insulin delivered above or below the patient's basal rate. So, a patient who only receives their basal rate will have an IOB of 0, regardless of how high or low is their basal rate. Their aIOB though will be positive and a higher basal rate will correspond to a higher aIOB. The aIOB metric can be used, for instance, to more accurately determine how much insulin should be provided to the patient 2014.

The aIOB metric may never reach zero because the patient 2014 may always have at least some small amount of insulin in their body. Accordingly, the aIOB metric may confuse patients used to seeing an IOB metric reach zero some amount of time after receiving an insulin bolus. Accordingly, the DAP system 2000 also uses the aforenoted “equivalent insulin on board” or “eqIOB” metric, which refers to a downshifted aIOB metric that includes the insulin information inherent in the aIOB metric while also eventually reaching zero due to the downshift. Presenting this eqIOB metric to the patient rather than the aIOB metric may therefore reduce confusion.

Responsible for estimating the aIOB metric is the aIOB estimator 2034 of the electronic device. After receiving a dose estimate from the pumping layer 2042 of the infusion pump, the aIOB estimator 2034 estimates the aIOB metric for the patient 2014 and provides the estimation to the eqIOB estimator 2032. The eqIOB estimator 2032 then translates the aIOB estimate into an eqIOB estimation and provides said estimation to the dose function selector 2020, discussed above, as well as the bolus calculator 2040. These components of the electronic device may use the eqIOB estimation in determining, respectively, which dosing function to select, and what bolus to provide to the patient.

As noted above, the transmitter 2018 of the CGM may transmit data regarding the patient to the receiver 2038 of the electronic device. This data may include, for instance, a five-minute reading (“FMR”) of the patient 2014. The receiver 2038 may then forward this data to the predictor 2036, which can thereby provide one or more predictors of future glucose values. The predictor 2036 may determine such predictor(s) by extrapolating a certain time period (e.g., 20 minutes) into the future from the present time via a least-squares linear fit to a certain number (e.g., four) most recent CGM sensor FMR readings received in the last specific time period of, e.g., 30 minutes. The predictor 2036 may provide the predictor(s) to the aIOB target selector 2022. The aIOB selected target from 2022 and the estimated aIOB from 2034 are processed at 2024, the output of which is processed by a multiplier at 2026 and further adjusted in accordance with a suitable maximum or minimum possible dose at 2028.

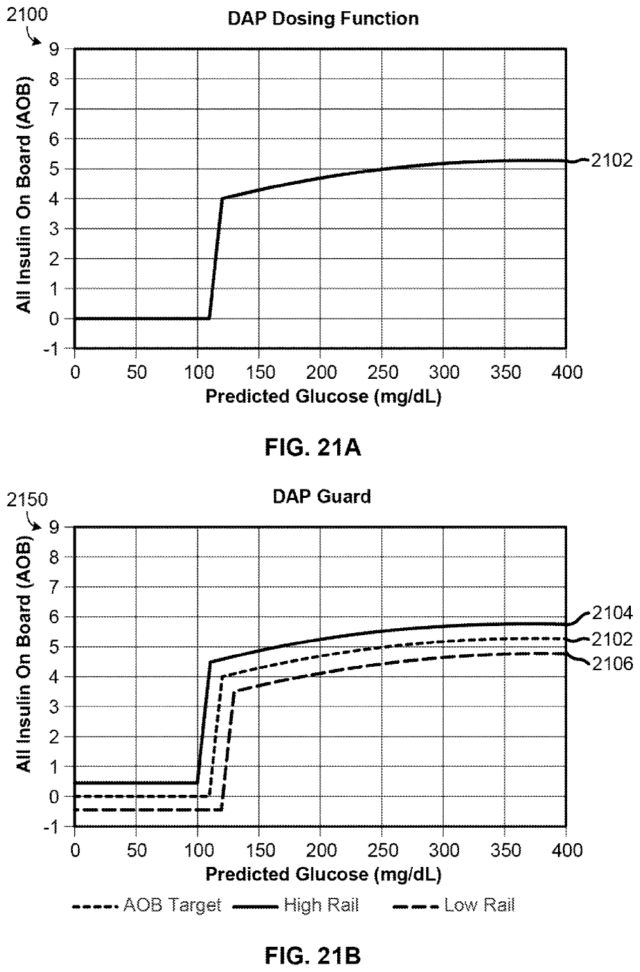

FIGS. 21A and 21B illustrate optimal aIOB predictions 2102 and guardrails 2104 and 2106 for the same (e.g., guardrails for predictions from a neural network, as discussed below), according to various embodiments of the present disclosure. The first graph 2100 illustrates a DAP dosing function, with an aIOB target 2102 as a function of predicted glucose. For example, as discussed above, a DAP system (e.g., DAP system 2000) can determine an optimal aIOB for a patient based in part on a blood glucose level of the patient. The DAP system can then provide insulin to the patient to bring the patient's IOB in line with the determined optimal aIOB.

The second graph 2150 illustrates example DAP guardrails 2104 and 2106 based on the optimal aIOB curve 2102 of the first graph 2100. For example, certain embodiments of the present invention may require guardrails, or boundaries, to ensure that optimal aIOB predictions from a neural network are not too far from expected aIOB values, as discussed in more detail hereinbelow. These guardrails may be established as percentages of the optimal aIOB curve 2102 (e.g., 90% and 110% of the curve), thus offering upper and/or lower bounds for aIOB predictions. Accordingly, predicted aIOB values can be compared against the guardrails 2104 and 2106 to determine whether the predictions are, respectively, too high or too low.

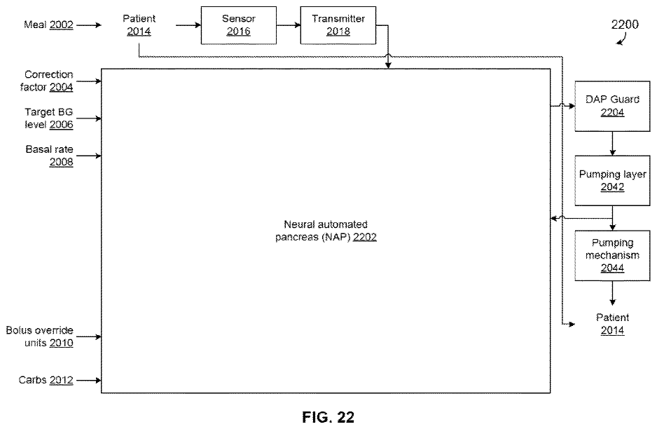

FIG. 22 illustrates another example system 2200 for closed- or semi-closed-loop operation of an infusion pump, according to various embodiments of the present disclosure. The example system 2200 is also referred to herein as a DAP Guard system 2200.

Like the DAP system 2000 of FIG. 20, the DAP Guard system 2200 includes a sensor 2016 and a transmitter 2018. Together, the sensor 2016 and the transmitter 2018 may constitute a CGM. The DAP Guard system 2200 also includes a pumping layer 2042 and a pumping mechanism 2044, which together may comprise an infusion pump (e.g., an ambulatory infusion pump).

However, unlike the DAP system 2000, in the illustrated implementation, the DAP Guard system 2200 includes a neural network, referred to herein as a neural automated pancreas (“NAP”) 2202. The NAP 2202 may perform similar functions as those performed by the electronic device discussed above with respect to the DAP system 2000. As illustrated, the DAP Guard system 2200 can receive various data, such as a correction factor 2004, a target blood glucose level 2006, a basal rate 2008, bolus override units 2010, and carbohydrates 2012. Additionally, the NAP 2202 can predict an optimal aIOB for the patient 2014 and can instruct an infusion pump that includes pumping layer 2042 and pumping mechanism 2044 to administer insulin to the patient 2014 accordingly. The target aIOB may suggest, for example, an amount of insulin that needs to be provided to the patient 2014. This can be determined, for example, by comparing the target aIOB to the current aIOB of the patient 2014. Accordingly, in some embodiments, the NAP 2202 provides a target aIOB to the infusion pump. However, in other embodiments, the NAP 2202 provides a dosing amount of insulin to the infusion pump.

The NAP 2202 may be less computationally intensive, for example, as compared to the instructions associated with the electronic device discussed above (e.g., selecting a dosing function, estimating an aIOB, selecting an aIOB target). Accordingly, the NAP 2202 may be more practical for portable devices, such as CGMs (or devices connected thereto), smartphones, or smartwatches. This may be especially true for CGMs, ambulatory infusion pumps, or portable devices connected thereto, which often have particular limits placed on their RAM and processor in order to meet battery-life and portability requirements.

In some embodiments, the NAP 2202 is a neural network trained using data from the DAP system 2000 discussed above with respect to FIG. 20. For example, the NAP 2202 can be trained on datasets that include the data provided to the DAP system 2000 (e.g., a correction factor 2004, a target blood glucose level 2006, a basal rate 2008, bolus override units 2010, and carbohydrates 2012) and corresponding predictions produced by the DAP system 2000, such as target aIOBs (also referred to as optimal aIOBs). With a sufficiently large dataset, the NAP 2202 may learn to predict target aIOBs based on new datasets that include, for instance, a correction factor 2004, a target blood glucose level 2006, a basal rate 2008, bolus override units 2010, and carbohydrates 2012.

A potential downside of the NAP 2202 is that the NAP 2202 may, at times, provide inaccurate estimations (e.g., of optimal aIOB). For example, given the significant number of estimations that the NAP 2202 may provide in a given day, a 91.34% accurate NAP 2202 may still provide 25 inaccurate estimations in a given day. Similarly, a 92.62% accurate NAP 2202 may provide 21 inaccurate estimations in a day. As another example, a 95.5% accurate NAP 2202 may provide 13 inaccurate estimations each day, and a 97.3% accurate NAP 2202 can provide as many as 8 inaccurate estimations daily. Even a 99.67% accurate NAP 2202 may still provide an inaccurate estimation on a daily basis (i.e., approximately 1 inaccurate estimation per day).

Accordingly, the DAP Guard system 2200 includes DAP Guard 2204 to detect and handle inaccurate estimations from the NAP 2202. The DAP Guard 2204 can include guardrails, or boundaries, for predictions provided by the NAP 2202 (see, e.g., guardrails 2104 and 2106 of FIG. 21B). As discussed above with respect to FIGS. 21A and 21B, these guardrails may be based on one or more optimal aIOB determinations (see, e.g., target aIOB curve 2102 of FIG. 21B) for a given blood glucose level.

If the prediction from the NAP 2202 is within the guardrails of the DAP Guard 2204 (e.g., less than a maximum threshold and/or greater than a minimum threshold), then the DAP Guard 2204 can send the prediction on to the pumping layer 2042 of the infusion pump. But if the prediction from the NAP 2202 does not satisfy the DAP Guard's 2204 guardrails, then the DAP Guard may, for example: (a) adjust the prediction to the nearest guardrail and send the adjusted prediction to the pumping layer 2042, (b) inform the pumping layer 2042 of the error and instruct the pumping layer 2042 to inform a user of the same, and/or (c) cause the infusion pump to administer a default amount of insulin for the current blood glucose level of the patient 2014, rather than administering insulin to the patient 2014 according to the predicted target aIOB of the NAP 2202.

Alternatively, or additionally, the DAP Guard 2204 can be implemented after the pumping layer 2042 (e.g., between the pumping layer 2042 and the pumping mechanism 2044). For example, in some embodiments, the pumping layer 2042 receives a target aIOB estimation from the NAP 2202, determines a dose estimate based on the target aIOB, and provides the dose estimate to the DAP Guard 2204. The DAP Guard 2204 may then determine whether the dose estimate is outside of bounds prescribed by guardrails. In this manner, the DAP Guard 2204 can be configured to determine whether a dose estimate satisfies a dose-estimate guardrail as an alternative to, or in addition to, determining whether an aIOB estimate satisfies an aIOB guardrail (as discussed above). Likewise, the DAP Guard 2204 can be further configured to review other parameters for delivery of a medicament (e.g., insulin) and determine whether said parameters are within boundaries prescribed by guardrails relating thereto (e.g., aIOB guardrails, dose estimate guardrails).

The DAP Guard approach to monitoring and correcting NAP 2202 predictions may similarly be less computationally intensive than other alternatives. For example, a k-nearest neighbors approach to neural network prediction correction may require more computational resources than are available on a portable device (e.g., a CGM, an ambulatory infusion pump). Accordingly, the DAP Guard 2204 can allow for use of a neural network without requiring a portable device implementing the neural network to connect to another device, such as a smartphone or a smartwatch, that may have access to more computational power. In this manner, the DAP Guard system 2200 can, in some embodiments, self-sufficiently determine how much insulin to provide to the patient 2014 and also correct the occasional improper determination.

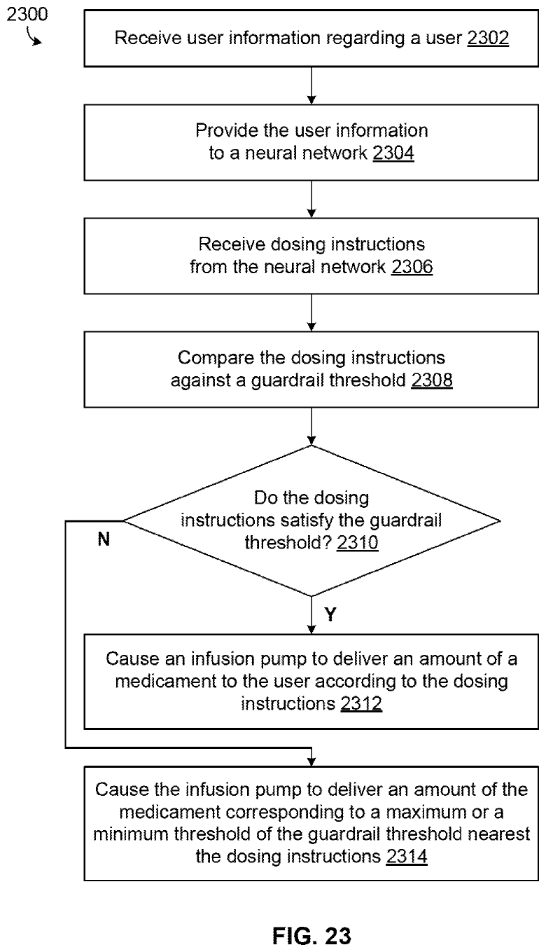

FIG. 23 is a flow chart of a method 2300 for neural-network-assisted operation of an infusion pump, according to various embodiments of the present disclosure. In some embodiments, the method 2300 is implemented by a CGM (incl., e.g., the sensor 2016, the transmitter 2018), an electronic device (incl., e.g., the dose function selector 2020, the aIOB target selector 2022, the eqIOB estimator 2032), a neural network (incl., e.g., NAP 2202), and/or an infusion pump (incl., e.g., pumping layer 2042, pumping mechanism 2046), such as those discussed above with respect to FIGS. 20 and 22.

For illustrative purposes, the following discussion refers to “a processor” as performing the operations of the method 2300. This processor may be a processor of the aforenoted CGM, electronic device, or infusion pump. Additionally, the processor may be a processor of a server, a mobile device, or any other electronic device discussed herein or otherwise known in the art. Moreover, it is noted that some of the operations of the method 2300 can be performed in an order other than the serial order suggested by the FIG. 23 and the following discussion. Likewise, some of the operations can be performed in parallel, and, in some embodiments, some of the operations need not be performed whatsoever.

In the illustrated embodiment, the processor receives (2302) user information regarding a user (e.g., patient 2014 of FIGS. 20 and 22). The user information may include dietary, physiological, and/or other information regarding the user. Specific examples of user information include a correction factor, a target blood glucose level, a current blood glucose level (e.g., detected by the sensor 2016 of FIG. 22), a basal rate, bolus override units, and an amount of carbohydrates consumed by the user. The user information may also include, for example, a blood glucose level of the user.

After receiving the user information, the processor provides (2304) the user information to a neural network (e.g., the NAP 2202 of FIG. 22). The neural network may have been trained in advance of performance of the method 2300, for example by providing training data to the neural network. The training data can include example correction factors, example target blood glucose levels, example current blood glucose levels, example basal rates, example bolus override units, example amounts of carbohydrates consumed by the user, and respective dosing instructions corresponding to the example correction factors, the example target blood glucose levels, the example current blood glucose levels, the example basal rates, the example bolus override units, and the example amounts of carbohydrates consumed by the user.

Responsive to providing the user information to the neural network, the processor receives (2306) dosing instructions from the neural network. As discussed above, the dosing instructions can include a target aIOB, a suggested amount of a medicament to deliver to the user, or another parameter for delivery of the medicament to the user (e.g., via the pumping mechanism 2044 of FIG. 22).

The processor then compares (2308) the dosing instructions against a guardrail threshold (see, e.g., DAP Guard 2204 of FIG. 22). For example, this may include comparing a metric included in the dosing instructions against a maximum threshold value (see, e.g., guardrail 2104 of FIG. 21) or a minimum threshold value (see, e.g., guardrail 2106 of FIG. 21). The processor compares the dosing instructions against the guardrail threshold to determine whether the dosing instructions satisfy the guardrail threshold. For instance, the dosing instructions may satisfy the guardrail threshold if the dosing instructions do not include any metrics outside of bounds prescribed by the guardrail threshold.

If the dosing instructions satisfy the guardrail threshold, then the processor causes (2312) an infusion pump (incl., e.g., the pumping layer 2042 and the pumping mechanism 2044 of FIG. 22) to deliver an amount of a medicament to the user according to the dosing instructions. However, if the dosing instructions do not satisfy the guardrail threshold, then the processor may cause (2314) the infusion pump to deliver another amount of the medicament. For example, the other amount of the medicament may correspond to a maximum or a minimum threshold of the guardrail threshold nearest the dosing instructions. In this manner, the processor can ensure that inaccurate estimations in the dosing instructions are kept within bounds and that the user still receives a proper amount of the medicament.

Alternatively, in some embodiments, the processor may forgo causing the infusion pump to deliver the medicament to the user (e.g., for a programmed or predetermined amount of time) if the dosing instructions do not satisfy the guardrail threshold. Instead, the processor may trigger an alert or an alarm or otherwise notify the user of the inaccurate estimation in the dosing instructions. In this manner, the processor can indicate to the user that manual intervention by the user may be necessary for delivery of a proper amount of the medicament.

In some embodiments, the processor is further configured to receive a deference indicator prior to comparing the dosing instructions against the guardrail threshold. The deference indicator may indicate to the processor a degree of deference that ought to be afforded to the dosing instructions from the neural network. For example, the processor may adjust or determine the guardrail threshold based on the deference indicator. If the guardrail threshold is a percentage of an expected aIOB metric (see. e.g., target aIOB curve 2102 of FIG. 21), then the deference indicator may include a percentage range outside of the expected aIOB metric where the dosing instructions will still be accepted (e.g., plus or minus 15%).

In some embodiments, the dosing instructions comprise an amount of the medicament to provide to the user, the guardrail threshold includes a maximum threshold, and determining that the dosing instructions satisfy the guardrail threshold includes determining that the amount of the medicament is less than or equal to the maximum threshold. Similarly, in some embodiments, the guardrail threshold includes a minimum threshold, and determining that the dosing instructions satisfy the guardrail threshold includes determining that the amount of the medicament is greater than or equal to the guardrail threshold.

In some embodiments, the processor is further configured to, prior to providing the user information to the neural network, receive from the infusion pump an amount of the medicament provided to the user. Accordingly, the user information provided to the neural network may include the amount of the medicament provided to the user.

Computing and other devices discussed herein can include memory. Memory can comprise volatile or non-volatile memory as required by the coupled computing device or processor to not only provide space to execute the instructions or algorithms, but to provide the space to store the instructions themselves. In one embodiment, volatile memory can include random access memory (RAM), dynamic random access memory (DRAM), or static random access memory (SRAM), for example. In one embodiment, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the disclosure.

In one embodiment, the system or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device.

An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations and should generally not be limited to any particular implementation exemplified herein, unless such limitations are expressly called out. In addition, an engine can itself be composed of more than one sub-engine, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. For example, in an embodiment, each of the processes depicted in FIG. 6 could be implemented within engines as described above. Likewise, in other contemplated embodiments, multiple defined functionalities may be implemented by a single engine that performs those multiple functions, possibly alongside other functions, or distributed differently among a set of engines than specifically illustrated in the examples herein.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that embodiments may comprise fewer features than illustrated in any individual embodiment described above. The embodiments described herein are not meant to be an exhaustive presentation of the ways in which the various features may be combined. Accordingly, the embodiments are not mutually exclusive combinations of features; rather, embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one embodiment,” “an embodiment,” or “some embodiments” means that a particular feature, structure, or characteristic, described in connection with the embodiment, is included in at least one embodiment of the teaching. The appearances of the phrase “in one embodiment” in various places in the specification are not necessarily all referring to the same embodiment.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein. For purposes of interpreting the claims, it is expressly intended that the provisions of Section 112, sixth paragraph of 35 U.S.C. are not to be invoked unless the specific terms “means for” or “step for” are recited in a claim.

1. A system for operation of an infusion pump, the system comprising:
a dosing function device configured to:
generate a predictive model to simulate effects of dosing decisions over time;
determine termination states based on the predictive model, each of the termination states representing an outcome following a sequence of dosing decisions over a finite time horizon;
apply a cost function to each dose in the sequence of dosing decisions such that each termination state has an associated cumulative cost; and
determine dosing functions, each of the dosing functions representing the cumulative cost associated with each termination state; and
an infusion pump configured to:
receive the dosing functions from the dosing function device;
select one of the dosing functions based on one or more characteristics of a user; and
deliver a dose of medicament to the user according to the selected dosing function.

2. The system of claim 1, wherein the predictive model comprises a simulation tree, and generating the predictive model comprises generating the simulation tree using a model-predictive-control (MPC).

3. The system of claim 2, wherein generating the dosing functions comprises performing a backward pass of the simulation tree.

4. The system of claim 1, wherein the one or more user characteristics comprise a target blood glucose, a basal rate, a correction factor, and a carb ratio.

5. The system of claim 4, wherein the one or more characteristics of the user are (i) received from a manual input or (ii) retrieved from a profile of the user.

6. The system of claim 1, wherein the infusion pump is further configured to, prior to delivering the dose of the medicament to the user, adjust the selected dosing function based on the one or more characteristics of the user.

7. The system of claim 1, wherein the infusion pump is further configured to, after delivering the dose of the medicament to the user according to the selected dosing function:
select another dosing function based on the one or more characteristics of the user; and
deliver another dose of the medicament to the user according to the other selected dosing function.

8. The system of claim 1, further comprising a continuous glucose monitor (CGM), and wherein:
the infusion pump is further configured to determine an optimal amount of the dose of the medicament based on the selected dosing function, a dosing history of the user, and data from the CGM; and
delivering the dose of the medicament to the user comprises delivering the optimal amount of the dose according to the selected dosing function.

9. The system of claim 8, wherein:
the infusion pump is further configured (i) to estimate an amount of UMI in the user, (ii) approximate an amount of insulin-on-board (IOB) in the user, or (iii) generate a glucose prediction for the user; and
wherein determining the optimal amount of the dose is further based on (i) the estimated amount of UMI, (ii) the approximated amount of IOB, or (iii) the generated glucose prediction.

10. The system of claim 1, wherein:
the infusion pump further comprises a memory; and
the infusion pump is further configured to, prior to delivering the dose of the medicament to the user, store a one-dimensional array in the memory, the one-dimensional array including IOB targets corresponding to respective glucose states.

11. An infusion pump comprising a processor and a non-transitory, computer-readable medium storing instructions which, when executed by the processor, cause the infusion pump to:
receive a set of fundamental dosing functions from a dosing function device, each of the fundamental dosing functions representing a cumulative cost associated with each of a plurality of termination states representing a respective outcome following a sequence of dosing decisions over a finite time horizon;
estimate a matching factor based on a basal rate and a correction factor;
select a fundamental dosing function from the set of fundamental dosing functions based on the matching factor and a target glucose;
estimate a scaling factor based at least on a basal rate of the selected fundamental dosing function;
determine a derived dosing function by multiplying the selected fundamental dosing function by the scaling factor; and
deliver a dose of medicament to a user according to the derived dosing function.

12. The infusion pump of claim 11, further comprising a display, and wherein the infusion pump is further configured to:
present the fundamental dosing functions and function parameters via the display;
receive a selected dosing function and selected adjustments for the selected dosing function; and
deliver a dose of medicament to the user according to the selected dosing function and the selected adjustments.

13. The infusion pump of claim 12, wherein the selected adjustments for the selected dosing function comprise adjustments to (i) a target glucose, (ii) a basal rate, (iii) a correction factor, (iv) or carb ratio.

14. The infusion pump of claim 11, wherein:
the infusion pump is further configured (i) to receive one or more characteristics of the user from a manual input or (ii) retrieve the one or more user characteristics from a profile of the user, the one or more user characteristics comprising a target blood glucose, the basal rate, a correction factor, and a carb ratio; and
selecting the fundamental dosing function is further based on the one or more user characteristics.

15. The infusion pump of claim 11, wherein the infusion pump is further configured to, after delivering the dose of the medicament to the user according to the derived dosing function:
select another fundamental dosing function from the set of fundamental dosing functions based on the matching factor and the target glucose;
deliver another dose of the medicament to the user according to the other selected fundamental dosing function.

16. A computer-implemented method for operation of an infusion pump, the method comprising:
receiving a set of fundamental dosing functions at the infusion pump and from a dosing function device, each fundamental dosing function representing a cumulative cost associated with each of a plurality of termination states representing a respective outcome following a sequence of dosing decisions over a finite time horizon;
estimating, at the infusion pump, a matching factor based on a basal rate and a correction factor;
selecting, at the infusion pump, a fundamental dosing function from the set of fundamental dosing functions based on the matching factor and a target glucose;
estimating, at the infusion pump, a scaling factor based at least on a basal rate of the selected fundamental dosing function;
determining, at the infusion pump, a derived dosing function by multiplying the selected fundamental dosing function by the scaling factor; and
delivering a dose of medicament to a user via the infusion pump and according to the derived dosing function.

17. The computer-implemented method of claim 16, wherein the infusion pump comprises a display and the method further comprises:
presenting, via the display, the fundamental dosing functions and policy parameters;
receiving, at the infusion pump, a selected dosing function and selected adjustments for the selected dosing function; and
delivering a dose of medicament to the user via the infusion pump and according to the selected dosing function and the selected adjustments.

18. The computer-implemented method of claim 17, wherein the selected adjustments for the selected dosing function comprise adjustments to (i) a target glucose, (ii) a basal rate, (iii) a correction factor, (iv) or carb ratio.

19. The computer-implemented method of claim 16, wherein:
the method further comprises (i) receiving, at the infusion pump, one or more characteristics of the user from a manual input or (ii) retrieving, at the infusion pump, the one or more user characteristics from a profile of the user, the one or more user characteristics comprising a target blood glucose, the basal rate, a correction factor, and a carb ratio; and
selecting the fundamental dosing function is further based on the one or more user characteristics.

20. The computer-implemented method of claim 16, wherein the method further comprises, after delivering the dose of the medicament to the user according to the derived dosing function:
selecting, at the infusion pump, another fundamental dosing function from the set of fundamental dosing functions based on the matching factor and the target glucose;
delivering another dose of the medicament to the user via the infusion pump and according to the other selected fundamental dosing function.

21-40. (canceled)
